(19) United States
(12) Patent Application Publication
(10) Pub. No.: US 2004/0078038 A1
(43) Pub. Date: Apr. 22, 2004

(54) Device for the electrothermal treatment of the human or animal body

(76) Inventors: Kai Desinger, Berlin (DE); Thomas Fricke, Berlin (DE); Thomas Stein, Berlin (DE)

Correspondence Address: HAHN LOESER & PARKS, LLP, TWIN OAKS ESTATE, 1225 W. MARKET STREET, AKRON, OH, 44313, US

(21) Appl. No.: 10/466,732

(22) PCT Filed: Jan. 17, 2002
(86) PCT No.: PCT/EP02/00459

(51) Int. Cl. A61B 18/14

(52) U.S. Cl. 606/50; 606/38; 606/40

(57) ABSTRACT

A device for electrothermal treatment of the human or animal body, in particular for tissue coagulation or electrotomy, has an elongate applicator with at least two electrodes for insertion into the body to be treated. To produce an electrical or electromagnetic field for heating the body tissue in the treatment region, the two electrodes are arranged on the applicator in electrically mutually insulated and mutually spaced relationship and are each connected by way of a respective feed line to an extracorporally arranged high-frequency generator, in order to be able better to control the treatment process there is provided a signal source which emits a signal that provides the user with information about the condition of the body tissue between the two electrodes during a treatment process.

The invention concerns a device for electrothermal treatment of the human or animal body, in particular for electrocoagulation or electrotomy, as set forth in the classifying portion of claim 1.

WO 97/17009 for example discloses a device of that kind, in which alternating currents in the frequency range of between about 300 kHz and some MHz are used for tissue coagulation and for tissue separation, whereby the treated tissue is coagulated or vaporised, this being referred to as electrocoagulation and electrotomy respectively. That procedure involves using a probe arrangement in which at least two electrodes are arranged in mutually spaced and insulated relationship on an elongate bar-shaped carrier and are supplied with the necessary HF-power with an extracorporally arranged high frequency generator so that produced between the electrodes is an adequate electrical or electromagnetic field which is limited to the area immediately surrounding the electrodes and coagulates or vaporises the body tissue which is between the two electrodes in the region of action of the electromagnetic and the resulting thermal field.

It has been found that the change in the electrical HF-impedance which can be measured between the electrodes, during the treatment procedure, that is to say during the tissue coagulation process, takes place in accordance with a pattern which remains substantially the same. While the absolute value of the HF-impedance to be measured between the electrodes depends on various influencing parameters such as for example the applicator geometry and the nature of the tissue, that HF-impedance has a typical configuration in respect of time which is characterised in that it rises sharply after a given treatment time, in which case the body tissue coagulates in the region of the electromagnetic field in question and as a result dries out, whereby the impedance of the individual tissue cells is substantially increased. That drying-out effect of the tissue cells advances rapidly until finally the electrodes are surrounded by dried-out tissue. Drying-out of the tissue and the resulting considerable increase in electrical HF-impedance in turn causes a collapse in the generator power due to mismatching. When the generator is switched off the impedance then falls exponentially to almost its minimum value because the input of power into the tissue is stopped and no further vaporisation of tissue fluid takes place. The consequence of this is that the dehydrated regions are filled with tissue water again and as a result the impedance correspondingly falls. If then the generator power is activated again after a certain time interval there is a fresh rise in impedance until the condition prior to the generator being switched off was achieved. That observed pattern in respect of electrical impedance between the two electrodes or the configuration in respect of time caused thereby in terms of the power delivered by the HF-generator can be useful as information so that the user can better manage the treatment process.

Therefore the object of the invention is to develop a device of the kind set forth in the opening part of this specification, in such a way that improved treatment and in particular more accurate determination of the treatment duration is possible.

According to the invention, in the device of the kind set forth in the opening part of this specification, that object is attained by a signal source which emits a signal which during a treatment process provides the user with information about the condition of the body tissue between the two electrodes.

The advantages of the invention are in particular that a signal is derived and made perceptible for the user, which informs the user during the treatment process about the condition in which the body tissue between the two electrodes is.

In a particularly preferred feature the signal source takes off from the HF-generator a value which is proportional to the emitted HF-current and also a further value which is proportional to the emitted HF-voltage. In accordance with the invention, the HF-power of the generator and/or the HF-impedance between the electrodes of the application arrangement is calculated from those two taken-off values and the signal emitted by the signal source depends on the calculated HF-power or the calculated electrical impedance so that the user is kept continuously informed during the treatment process by way of the HF-impedance which is measured between the two electrodes and which is an integral measurement in respect of the progress of tissue coagulation.

Particularly preferably the signal source emits a characteristic signal if the electrical HF-impedance between the two electrodes exceeds a predetermined value at which tissue coagulation has come to a conclusion in the region of the electrodes so that then the user can move the applicator into a different local position within the body tissue or can terminate the procedure.

In a particularly preferred feature the signal source is an acoustic signal source which emits an audible signal. The frequency of the emitted signal preferably depends on the configuration in respect of time of the emitted HF-power from the HF-generator or alternatively the configuration in respect of time of the HF-impedance which is present between the two electrodes. In this embodiment of the invention the user can recognise the change in the tissue on the basis of the pitch of the sound. If the frequency of the audible signal also increasingly increases in the HF-impedance of the body tissue and the signal goes over to the characteristic signal when the impedance of the body tissue between the two electrodes exceeds a predetermined value or the power delivery of the HF-generator falls below a predetermined value. The characteristic signal can be an acoustic signal of constant frequency, alternatively it can also be in the form of an audible signal which is modulated in respect of time, for example an audible signal in pulse form, which strikingly indicates to the user that the predetermined impedance limit has been exceeded.

In accordance with a further preferred embodiment of the invention the signal source emits a shut-down signal which shuts down the HF-generator or separates it from the electrodes if the electrical impedance between the electrodes exceeds the predetermined threshold value or the corresponding emitted HF-power of the generator falls below the predetermined value.

Advantageous developments of the invention are characterised by the features of the appendant claims.

An embodiment of the invention is described in greater detail hereinafter with reference to the drawings in which:

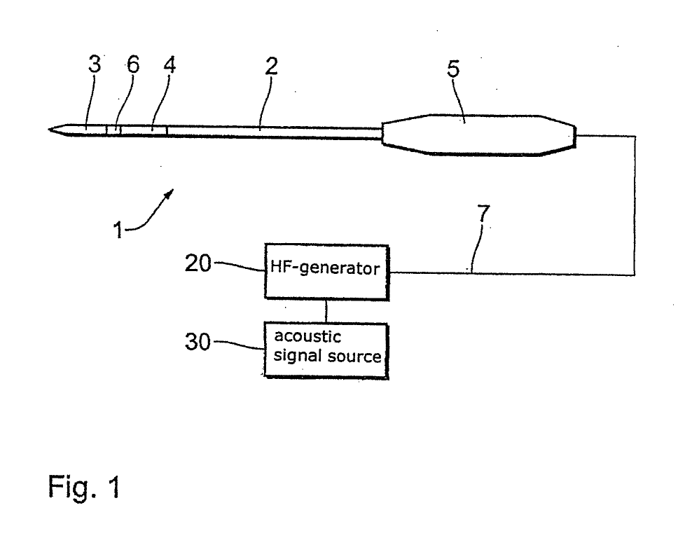

FIG. 1 is a diagrammatic view of the device,

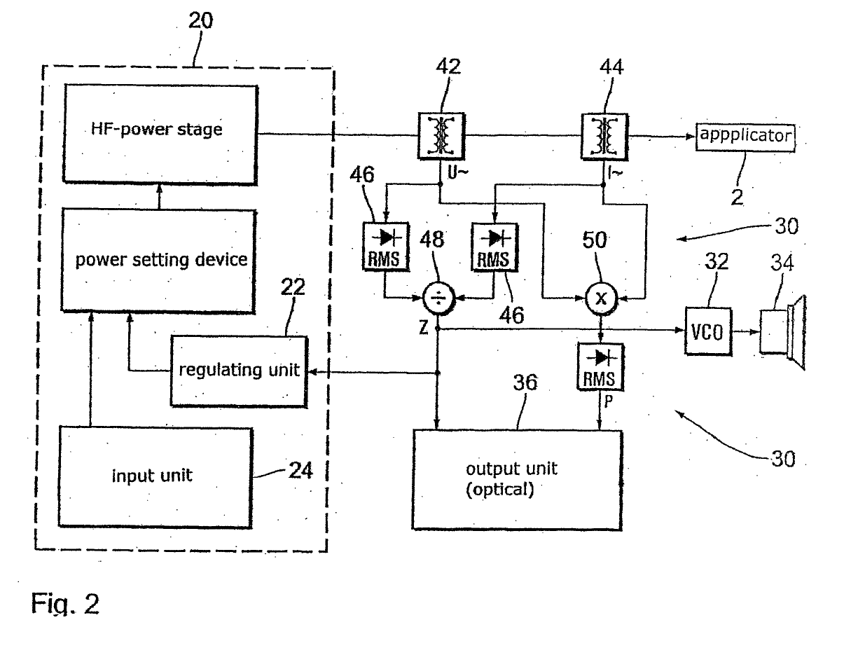

FIG. 2 shows a schematic circuit diagram of the device,

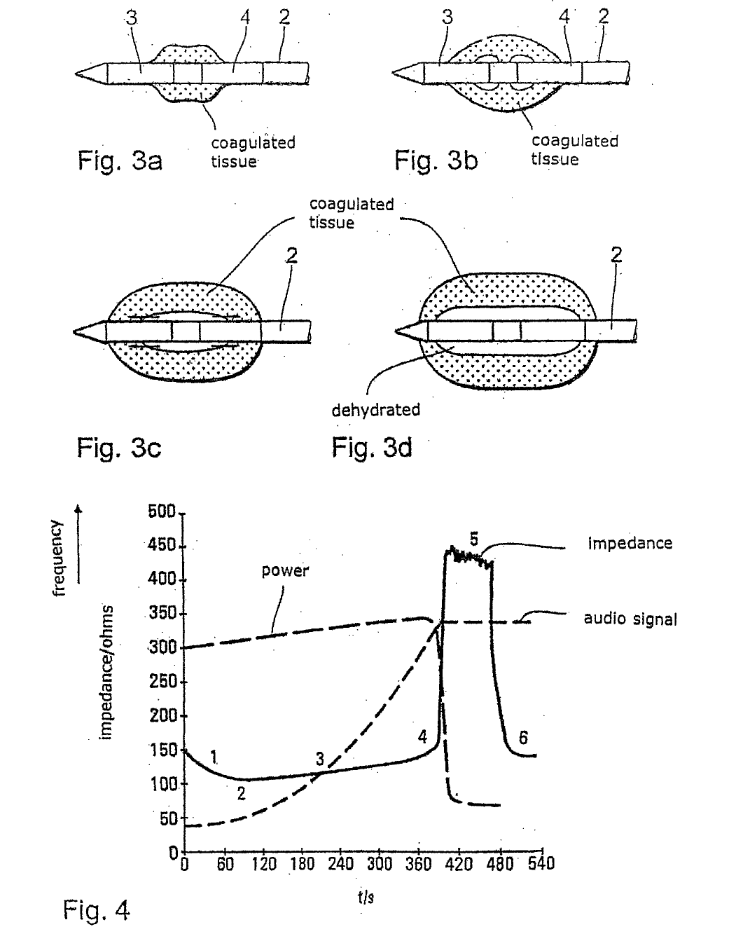

FIG. 3 shows a diagrammatic view of a coagulation process during a treatment procedure, and

FIG. 4 shows the diagrammatic configuration of the HF-impedance, between the electrodes of the applicator, of the body tissue during a treatment procedure.

FIG. 1 shows a device for electrothermal treatment of the human or animal body, which includes an applicator 1 which has an electrically insulated shank 2 and an electrode portion comprising a distal electrode 3 which tapers to a point at the free end and a proximal electrode 4. The electrodes 3 and 4 form a component part of the applicator 1 and are separated from each other by an insulating spacer element 6. Adjoining the proximal end of the shank is the handle with the electrical feed line 7. The electrodes 3 and 4 are connected by way of the feed line 7 to the HF-generator 20. The applicator 1 can be held by the user at a grip portion 5 and guided during the treatment procedure.

The HF-generator 20 is provided with an acoustic signal source 30 which, during a treatment process, takes off or senses the electrical output parameters from the HF-generator and produces a signal which gives the user information about the condition of the body tissue which is between the two electrodes in the therapy region.

As can be seen in particular from FIG. 2 voltage and current of HF-power passed from the HF-generator 20 to the applicator 1 is taken off by way of the coupling member 42 and the coupling member 44 and ascertained from those measurement parameters, by way of rectifier stages 46, by means of a dividing member 48, is the electrical impedance which arises out of the voltage applied across the two electrodes 3, 4 and the current flowing between the electrodes 3, 4 and which therefore represents information about the condition of the body tissue between the two electrodes. In addition, the HF-power which is introduced into the therapy region by way of the applicator is obtained by way of a multiplier member 50.

A signal which is derived from the calculated electrical impedance z and which is for example proportional is then fed by way of an electrical circuit 32 to a loudspeaker 34 which emits an acoustic audible signal whose frequency depends on the electrical HF-impedance of the body tissue between the electrodes 3, 4. For example the frequency of the signal emitted by the loudspeaker 34 increases if the impedance increases. If the impedance z exceeds a predetermined threshold value the acoustic signal changes into a sound signal at constant frequency which as a characteristic signal indicates to the doctor carrying out the treatment that the body tissue between the electrodes 3, 4 is coagulated and is dehydrated to a considerable degree so that the treatment ends at the treatment location in question and the applicator 1 can therefore either be displaced to another treatment location or withdrawn from the body tissue.

The sensing devices 42, 44, the rectifier circuits 46, the dividing member 48 and optionally the multiplier member 50 together with the electrical circuit 32 and the loudspeaker 34 represent the signal source 30 to which in the illustrated embodiment there is also added an optical output unit 36 which can display the electrical HF-impedance between the electrodes 2, 3 and/or the power P delivered to the electrodes 2, 3 and/or—instead of the acoustic audible signal—an optical information signal for the user, which can depend on the impedance between the electrodes 3, 4 and/or the power P delivered at the electrodes.

As shown in FIG. 2, derived from the output signal of the signal source 30 which depends on the emitted HF-power of the generator or the electrical HF-impedance of the body tissue between the electrodes 3, 4 is a regulating signal which is returned to the HF-generator 20 and is there processed in a regulating unit 22 in such a way that it then serves to regulate the HF-power delivered. For example the regulating unit 22 in the generator 20 can serve to produce a control signal as soon as the impedance, after a preceding rise in impedance, falls again below a predetermined absolute or relative value which activates the generator 20 again in order thereby to proceed with the coagulation process. Alternatively in the regulating procedure a given impedance value can be predetermined as the reference value and the power delivered by the HF-generator 20 can be regulated in such a way that the actual impedance value (actual value) approaches the predetermined impedance value (reference or target value) in the desired manner continuously or in intervals.

In a preferred embodiment of the invention the input unit 24 of the HF-generator 20 makes it possible to predetermine a power/time profile which is based on the delivered power of the HF-generator 20.

FIGS. 3a through 3d show the body tissue in the region of the electrodes 3, 4 of the applicator 2 in the course of a progressive advance in treatment in respect of time, and thus an increase in the coagulated area of tissue. The coagulation process begins in the adjoining body tissue which is disposed in the region of the mutually adjacent zones of the electrodes 3, 4. The area of coagulation then spreads forwardly towards the free tip of the applicator 2 and proximally to the proximal end of the electrode 4, see also the arrows in FIG. 3c. With an increasing treatment time, a dehydrated zone is then produced in the immediate proximity of the electrodes, that zone finally extending over the length of both electrodes 3, 4. The formation of the dehydrated tissue zone around the electrodes 3, 4 involves a considerable increase in electrical impedance z which is measured between the electrodes 3, 4. The impedance reaches its maximum when the configuration shown in FIG. 3d applies, as then virtually the entire zone between the two electrodes is formed by a dehydrated zone. That extreme increase in impedance causes a great reduction in the delivered generator power by virtue of mismatching which is caused by the increase in impedance. The configuration of the impedance gives information about the progress of the coagulation process in the surrounding tissue. It can be seen from the impedance configuration when the applicator 2 has reached the coagulation volume that is the maximum that it can generate—in dependence on the applicator structure and the HF-power.

As can be seen in particular from FIG. 4 the impedance rises strongly at point 4 on the impedance curve to a maximum value at the location 5. The audio signal which is produced by the signal source 30 and emitted by way of a loudspeaker 34— in the view in FIG. 4—steadily increases in frequency and then, when the impedance of the body tissue between the electrodes 3, 4 rises above a predetermined threshold value, goes to a constant frequency which indicates to the doctor conducting the treatment that the maximum coagulation volume which can be generated has been reached, and the generator can either be switched off or the applicator 2 can be moved into another treatment position. When the generator 2 is switched off the impedance very rapidly falls to its minimum value again because the input of power into the tissue is stopped and vaporisation of tissue water no longer takes place, but rather the dehydrated regions of tissue fill up again with tissue water, see point 6 of the impedance configuration. If then the generator is switched on once again and the coagulation process continued then the impedance very quickly rises again to its maximum value which is characterised by dehydration of the region of tissue through which the electromagnetic field passes.

1. A device for electrothermal treatment of the human or animal body, in particular for tissue coagulation or electrotomy, comprising an elongate applicator (1) having at least two electrodes (3, 4) for insertion into the body to be treated, wherein to produce an electrical or electromagnetic field for heating the body tissue in the treatment region the two electrodes (3, 4) are arranged on the applicator in electrically mutually insulated and mutually spaced relationship and are each connected by way of a respective feed line to an extracorporally arranged high-frequency generator (20),
characterised by a signal source (30) which emits a signal which during a treatment process provides the user with information about the condition of the body tissue between the two electrodes (3, 4).

2. A device as set forth in claim 1
characterised in that during a treatment process the signal source (30) takes off from the HF-generator parameters proportional to the delivered HF-current and to the delivered HF-voltage, the delivered HF-power of the generator (20) and/or the HF-impedance between the electrodes (3, 4) is calculated from those parameters and a signal is emitted which depends on the calculated delivered HF-power of the generator (20) and/or the calculated electrical HF-impedance which the body tissue between the two electrodes (3, 4) has.

3. A device as set forth in claim 2
characterised in that the signal source emits a characteristic signal when the electrical HF-impedance which the body tissue between the two electrodes (3, 4) has during a treatment process exceeds a predetermined value.

4. A device as set forth in claim 1 or claim 2
characterised in that the signal source (30) emits a characteristic signal when the delivered HF-power of the HF-generator (20) falls below a predetermined value during a treatment process.

5. A device as set forth in one of the preceding claims
characterised in that the signal source (30) is an acoustic signal source which emits an audible signal whose frequency depends on the configuration in respect of time of the delivered HF-power of the HF-generator (20) or the configuration in respect of time of the HF-impedance between the electrodes (3, 4).

6. A device as set forth in one of claims 1 through 4
characterised in that the signal source (30) is an optical signal source which emits a visible signal whose frequency in respect of time depends on the configuration in respect of time of the delivered HF-power of the HF-generator (20) or the configuration in respect of time of the HF-impedance between the electrodes (3, 4).

7. A device as set forth in claim 1 or claim 2
characterised in that a signal for regulation of one of the electrical output parameters of the HF-generator (20) is derived from the signal of the signal source (30) which depends on the delivered HF-power of the HF-generator (20) or the electrical HF-impedance between the electrodes (3, 4), and is returned to the HF-generator (20).

8. A device as set forth in one of claims 1 through 7
characterised in that the frequency of the signal emitted by the signal source (30) increases when the impedance of the body tissue between the electrodes (3, 4) increases during a treatment process.

9. A device as set forth in one of claims 1 through 8
characterised in that the frequency of the signal emitted by the signal source (30) decreases when the impedance of the body tissue between the two electrodes (3, 4) increases.

10. A device as set forth in one of the preceding claims
characterised in that the frequency of the signal emitted by the signal source (30) is approximately proportional to the impedance of the body tissue between the electrodes (3, 4) or inversely proportional to the delivered HF-power of the HF-generator (20).

11. A device as set forth in one of the preceding claims
characterised in that the audible signal emitted by the signal source (30) suddenly changes when the impedance of the body tissue between the electrodes (3, 4) during a treatment process rises above a predetermined value or when the power delivery from the HF-generator (20) falls below a predetermined value.

12. A device as set forth in one of the preceding claims
characterised in that the audible signal emitted by the signal source (30) is cycled when the impedance of the body tissue between the electrodes (3, 4) during a treatment process rises above a predetermined value or when the power delivery from the HF-generator (20) falls below a predetermined value.

13. A device as set forth in one of the preceding claims
characterised in that the acoustic or optical signal source (30) is contained in the HF-generator (20).

14. A device as set forth in one of the preceding claims
characterised in that the acoustic or optical signal source (30) feeds a switch-off signal to the HF-generator (20) which separates the HF-generator (20) from the electrodes (3, 4) or switches it off when the electrical impedance of the body tissue between the electrodes (3, 4) during a treatment process exceeds a predetermined absolute or relative value.

15. A device as set forth in one of the preceding claims
characterised in that the signal source (30) feeds to the HF-generator (20) a signal which activates the HF-generator (20) again or connects it to the electrodes (3, 4) when the impedance of the body tissue between the electrodes (3, 4), after a preceding rise in impedance, falls again below a predetermined absolute or relative value.

16. A device as set forth in one of claims 7 through 15
characterised in that the delivered power from the HF-generator (20) is regulated in such a way that upon a rise in the impedance value it is prevented from exceeding a predetermined value.

17. A device as set forth in one of the preceding claims
characterised in that the delivered power from the HF-generator (20) is controlled in accordance with a time/power profile which can be predetermined by the user.

18. A device as set forth in one of the preceding claims
characterised in that the elongate applicator (2) which carries the electrodes (3, 4) is in the form of a bar of a constant or variable cross-section and goes to a point at its free front end.
